(19) United States
(12) Patent Application Publication
(10) Pub. No.: US 2011/0250692 A1
(43) Pub. Date: Oct. 13, 2011

(54) METHOD FOR PRODUCING INDUCED PLURIPOTENT STEM CELLS

(75) Inventors: Shinya Yamanaka, Kyoto (JP); Koji Tanabe, Kyoto (JP)

(73) Assignee: KYOTO UNIVERSITY, KYOTO (JP)

(21) Appl. No.: 13/059,188

(22) PCT Filed: Oct. 30, 2009
(86) PCT No.: PCT/JP09/69015

§ 371 Date: Feb. 15, 2011

Related U.S. Application Data
(60) Provisional application No. 61/193,122, filed on Oct. 30, 2008.
(60) Provisional application No. 61/202,385, filed on Feb. 24, 2009.

(51) Int. Cl. C12N 5/071; C12N 15/85

(52) U.S. Cl. 435/455; 435/377; 435/363; 435/354; 435/352; 435/366

(57) ABSTRACT

The present invention relates to a method for producing mammalian induced pluripotent stem cells, comprising introducing mammal-derived reprogramming factors comprising Oct3/4 and Nanog, or nucleic acids encoding Oct3/4 and Nanog, into mammal-derived somatic cells and thereby inducing induced pluripotent stem cells from the somatic cells, wherein the reprogramming factors comprise neither Sox2 nor nucleic acid encoding Sox2.

### FIELD OF INVENTION

The present invention relates to a novel method for producing induced pluripotent stem cells. Specifically, the present invention relates to a method for producing induced pluripotent stem cells from somatic cells, which is characterized by using Oct3/4 and Nanog, or nucleic acids encoding them, as major reprogramming factors.

Hereinafter, the term “induced pluripotent stem cells” may be simply referred to as “iPS cells.”

### BACKGROUND OF INVENTION

In recent years, mouse and human iPS cells have been established in succession. Takahashi and Yamanaka (1) have established iPS cells by introducing Oct3/4, Sox2, Klf4 and c-Myc genes into reporter mouse-derived fibroblasts containing a neomycin resistance gene knocked in the Fbx15 gene locus to cause forced expression of the genes. Okita et al (2) have succeeded in establishment of iPS cells (Nanog iPS cells) almost equivalent to embryonic stem (ES) cells in terms of gene expression and epigenetic modification by producing transgenic mice in which a green fluorescent protein (GFP) gene and a puromycin resistance gene have been integrated into the Nanog gene locus, the expression of which is limited to pluripotent cells as compared with Fbx15, causing forced expression of the above four genes in the fibroblasts from the mouse, and then selecting puromycin-resistant and GFP positive cells. Other groups have also reproduced similar results (3 and 4). Thereafter, it has been revealed that iPS cells can also be produced using 3 factors excluding a c-Myc gene (5).

Furthermore, Takahashi et al (6) have succeeded in establishment of iPS cells by introducing 4 genes into human dermal fibroblasts as in the case of mice. Meanwhile, Yu et al (7) have produced human iPS cells using Oct3/4, Sox2, Nanog, and Lin28 genes. As described above, it has been demonstrated that iPS cells equivalent to ES cells in terms of pluripotency can be produced in humans and mice by introducing specific factors into somatic cells.

Yu et al (7) above have obtained iPS cell colonies by introducing 3 out of 4 types of gene excluding Nanog or Lin28 gene, but they have not obtained any iPS cell colonies via introduction of 3 types of gene excluding Oct3/4 or Sox2 gene. This suggests the importance of the Oct3/4 and Sox2 genes in establishment of iPS cells (see also (8)). Moreover, Park et al (9) have also mentioned that Oct3/4 and Sox2 are essential for establishment of iPS cells.

Furthermore, patent document (10) describes production of mouse iPS cells via introduction of Oct3/4, Sox2, Klf4, and c-Myc genes and discloses a technique which can induce iPS cells in a Dox-inducible system. Patent document (10) also describes successful induction of iPS cells with the use of mature B cells as somatic cells.

Moreover, the patent documents of Yamanaka et al (11) and Thomson et al (8) have been published with respect to the induction of iPS cells from somatic cells.

### REFERENCES

1. Takahashi, K. and Yamanaka, S., Cell, 126: 663-676 (2006)
2. Okita, K. et al., Nature, 448: 313-317 (2007)
3. Wernig, M. et al., Nature, 448: 318-324 (2007)
4. Maherali, N. et al., Cell Stem Cell, 1: 55-70 (2007)
5. Nakagawa, M. et al., Nat. Biotethnol., 26: 101-106 (2008)
6. Takahashi, K. et al., Cell, 131: 861-872 (2007)
7. Yu, J. et al., Science, 318: 1917-1920 (2007)
8. WO2008/118820
9. Park I. H et al., Nature, 451: 141-146 (2008)
10. WO2008/124133
11. WO2007/069666

### SUMMARY OF INVENTION

### Problem to be Solved by Invention

Since Based on the above-described discovery of Yamanaka et al., (Takahashi, K. and Yamanaka, S. Cell 126: 663-676 (2006)) as a breakthrough, several combinations of reprogramming factors that make it possible to induce induced pluripotent stem cells from somatic cells have been proposed. With any combination, induced pluripotent stem cells are induced from somatic cells. A common reprogramming factor among the proposed combinations of reprogramming factors is Oct3/4 alone. Recently, it has been reported that reprogramming takes place with the use of Oct3/4 alone. This case can be said to be a special example since somatic cells used herein are neural stem cells in which an Sox2 gene is endogenously expressed (Kim, J. B. et al., Cell 136: 411-419 (2009)). Also, among combinations of Oct3/4 with other reprogramming factors, a combination of 2 factors has been reported, which is a combination of Oct3/4 and Sox2 (WO2008/118820), a combination of Oct3/4 and Klf4 (Kim, J. B. et al., Nature 454: 646-650 (2008), Shi, Y. et al., Cell Stem Cell, 2: 525-528 (2008)), or a combination of Oct3/4 and c-Myc (Kim, J. B. et al., Nature 454: 646-650 (2008)).

However, it has not yet been revealed why the reported combinations of reprogramming factors make it possible to induce somatic cells into induced pluripotent stem cells. Nor has a mechanism of the induction been clarified.

Under these circumstances, an object of the present invention is to find a novel combination of reprogramming factors, which has never been reported and enables reprogramming of somatic cells, and thus to provide a method for producing induced pluripotent stem cells from somatic cells using such factors in combination.

### Means to Solve Problem

International Publication WO2008/118820 (published Oct. 2, 2008) describes combinations of reprogramming factors for reprogramming somatic cells into induced pluripotent stem cells, wherein the essential reprogramming factors are Oct3/4 and Sox2, to which at least one reprogramming factor of Nanog and Lin28 can be added. According to this document, examples of combinations of reprogramming factors are: Oct3/4 and Sox2; Oct3/4, Sox2, and Nanog; Oct3/4, Sox2, and Lin28; and Oct3/4, Sox2, Nanog, and Lin28. The document also demonstrates that in contrast to such examples, a combination of Oct3/4, Nanog, and Lin28 results in no reprogramming. Furthermore, Huangfu D. et al., Nature Biotech., Vol. 26, No. 11: 1269-1275 (2008) (online-published Oct. 12, 2008) describes an attempt to establish induced pluripotent stem cells via introduction of two factors (from among Oct3/4, Sox2, and K1f4) and VPA into human fibroblasts, and the failure to establish induced pluripotent stem cells with the use of combinations other than a combination of Oct3/4 and Sox2.

The present inventors have now found that reprogramming of somatic cells into induced pluripotent stem cells is possible, surprisingly, with a combination of only Oct3/4 and Nanog. The combinations of two reprogramming factors that have been reported to date are only Oct3/4 and Sox2 (International Publication WO2008/118820), Oct3/4 and Klf4 (Kim, J. B. et al., Nature 454: 646-650 (2008), Shi, Y. et al., Cell Stem Cell, 2: 525-528 (2008)), and Oct3/4 and c-Myc (Kim, J. B. et al., Nature 454: 646-650 (2008)). However, in the case of the latter two combinations (Nature 454: 646-650 (2008) and Cell Stem Cell, 2: 525-528 (2008)), the somatic cells are mouse neural stem cells in which Sox2 gene is originally expressed. Hence, it is not necessary to add the Sox2 gene as a reprogramming factor. Therefore, in contrast to the conventional finding that Oct3/4 and Sox2 are essential, the combination of two reprogramming factors of the present invention, i.e., Oct3/4 and Nanog, is a surprising combination completely unpredictable by a person skilled in the art. In addition, the adult human dermal fibroblasts (HDF) that the present inventors have used in experiments are cells that do not express Sox2 (Cell, 131, 861-872 (2007)). Even from this viewpoint, the combination of two reprogramming factors consisting of Oct3/4 and Nanog in the present invention is a surprising combination completely unpredictable by a person skilled in the art.

Hence, the present invention has the following characteristics.

The present invention provides a method for producing mammalian induced pluripotent stem cells, which comprises introducing mammal-derived reprogramming factors comprising Oct3/4 and Nanog, or nucleic acids encoding Oct3/4 and Nanog, into mammal-derived somatic cells and thereby inducing induced pluripotent stem cells from the somatic cells, wherein the reprogramming factors comprise neither Sox2 nor nucleic acid encoding Sox2.

According to an embodiment thereof, the above reprogramming factors further comprise Lin28 or a nucleic acid encoding Lin28.

According to another embodiment, the above reprogramming factors consist of Oct3/4 and Nanog, or nucleic acids encoding Oct3/4 and Nanog.

Alternatively, according to another embodiment, the above reprogramming factors consist of Oct3/4, Nanog and Lin28, or nucleic acids encoding Oct3/4, Nanog, and Lin28.

According to another embodiment, mammals from which the above reprogramming factors and somatic cells are derived are the same or different from each other.

According to another embodiment, the above mammals are primates or rodents.

According to another embodiment, the above primates are humans.

According to another embodiment, the above rodents are mice.

According to still another embodiment, the above nucleic acids are contained in a vector.

According to another embodiment, the above nucleic acids are integrated into the genome of the above somatic cells or exist within the cells in the state not integrated into the genome.

According to still another embodiment, the induction of the above induced pluripotent stem cells is carried out in the presence of a substance for improving an efficiency of establishment of the cells.

According to another embodiment, the above substance for improving an efficiency of establishment is a histone deacetylase (HDAC) inhibitor, a histone methyltransferase (G9a) inhibitor, or a DNA methylase (Dnmt) inhibitor.

### BRIEF DESCRIPTION OF DRAWINGS

FIG. 1 shows photographs showing the morphology of the established iPS cell clone T4F-1. The left photograph shows the image of a colony upon its formation and the right photograph shows the image of a colony of passage 3.

FIG. 2 shows a photograph showing the results of Genomic-PCR analysis performed for established human iPS cell clones. In this figure, “T4F-1” denotes a clone with the only two genes, Oct3/4 and Nanog, inserted into the genome, and “T4F-2” denotes a clone with the four genes, Oct3/4, Sox2, Nanog and Lin28, inserted into the genome. Further, “Y4F” denotes a clone established with the introduction of four genes, Oct3/4, Sox2, Klf4, and c-Myc, and “Y3F” denotes a clone established with the introduction of three genes, Oct3/4, Sox2, and Klf4. “HDF” denotes adult human dermal fibroblasts used for introduction and “H2O” denotes a solvent (water) as a negative control.

FIG. 3 shows a RT-PCR photograph showing established human iPS cell clones expressing ES cell-specific marker genes (Oct3/4, Nanog, Lin28, PH34, Dnmt3b, and Nodal). In this figure, “T4F-1” and “T4F-2,” denote established clones, and both “201B6 P19” and “201B7 P37” denote iPS cell clones established using Oct3/4, Sox2, Klf4, and Nanog as described in Takahashi, K. et al., Cell, 131: 861-872 (2007) (wherein P19 or P37 is the number of passages). “ES” is ES cell, “AHDF” is adult human dermal fibroblast used for introduction, and “H2O” is a negative control.

FIG. 4 shows images of the histological staining (hematotoxin-eosin staining) of the teratomas that were obtained by injecting the human iPS cell clone (T4F-1), which was established by insertion of two genes (Oct3/4 and Nanog) into the genome, into the testis of a Scid mouse. This figure shows, from the left, the histological images of neural tube tissue, cartilage tissue and intestine-like epithelial structure.

FIG. 5 shows photographs showing the morphologies of the iPS cell clones, ON-1 and ON-5, which were established by introduction of two genes (Oct3/4 and Nanog). The left photographs show the images of colonies upon their formation and the right photographs show the images of colonies of passage 3.

FIG. 6 shows a RT-PCR photograph showing the established human iPS cell clones expressing ES cell-specific marker genes (Oct3/4, Nanog, Lin28, PH34, and Dnmt3b). In this figure, “ON-1 to ON-6” denote clones each established by introduction of two genes, and “201B7 P 19” (P19 is the number of passages) denotes an iPS cell clone established with the use of Oct3/4, Sox2, Klf4, and Nanog as described in Takahashi, K. et al., Cell, 131: 861-872 (2007). “AHDF” is adult human dermal fibroblast used for introduction, and “H2O” is a negative control.

FIG. 7 shows photographs showing the morphologies of iPS cell clones, ONLM (FIG. 7A) and ONL (FIG. 7B), established by introduction of four genes (Oct3/4, Nanog, Lin28, and c-Myc) and three genes (Oct3/4, Nanog, and Lin28), respectively, into adult human dermal fibroblasts, and an iPS cell clone ON (FIG. 7C) established by introduction of two genes (Oct3/4 and Nanog) into neonatal human foreskin fibroblasts. All the left photographs show images of colonies that were formed. The center and the right photographs show the images of colonies of passage 1 or passage 10.

FIG. 8 shows photographs showing the morphology of an iPS cell clone established by introduction of two genes (Oct3/4 and Nanog) into the human dental pulp stem cells DP31. The left photograph shows the image of a colony upon its formation, and the right photograph shows the image of colonies of passage 2.

FIG. 9 shows a photograph showing the results of Genomic-PCR analysis performed for established human iPS cell clones. This figure shows, from the left lane, the results of the following cells.
ONL: iPS cell clone established by introduction of three genes, Oct3/4, Nanog, and Lin28, into human adult dermal fibroblasts
AHDF-ON: iPS cell clone established by introduction of two genes, Oct3/4 and Nanog, into human adult dermal fibroblasts
NHDF-ON: iPS cell clone established by introduction of two genes, Oct3/4 and Nanog, into human neonatal foreskin fibroblasts.
201B6: iPS cell clone established with the use of Oct3/4, Sox2, Klf4, and Nanog as described in Takahashi, K. et al., Cell, 131: 861-872 (2007).
AHDF: human adult dermal fibroblasts
NHDF: human neonatal foreskin fibroblasts

FIG. 10 shows a photograph showing the results of Genomic-PCR analysis performed for established human iPS cell clones. FIG. 10 shows, from the left lane, the results of the following cells.
OSKMNL: iPS cell clone established by introduction of six genes, Oct3/4, Sox2, Klf4, c-Myc, Nanog and Lin28, into human adult dermal fibroblasts
Dp31 ON: iPS cell clone established by introduction of two genes, Oct3/4 and Nanog, into human dental pulp stem cells Dp31
Dp31: human dental pulp stem cells
H2O: negative control (water)

FIG. 11 shows histological staining (hematotoxin-eosin staining) images of the teratomas that were obtained by injecting the human iPS cell clone established by introduction of two genes, Oct3/4 and Nanog, into human adult dermal fibroblasts, into the testis of a Scid mouse. This figure shows, from the left, the images of a nervous tissue, a glandular epithelial tissue, and a cartilage tissue.

FIG. 12 shows photographs showing the results of immunostaining, wherein human iPS cell clones, which were established by introduction of two genes, Oct3/4 and Nanog, into human adult dermal fibroblasts, expressed stem cell markers.

FIG. 13 shows photographs showing the results of confirming that a human iPS cell clone established by introduction of two genes, Oct3/4 and Nanog, into human adult dermal fibroblasts has an ability to differentiate into three germ layers. Specifically, this was confirmed by staining with respective antibodies to βIII-tubulin (“Tubrin”), α-fetoprotein (“AFP”), Vimentin, smooth muscle actin (“SMA”), and Desmin. Cy3-Nega and 488-Nega were negative controls.

### MODES FOR CARRYING OUT INVENTION

The present invention will be further described in detail.

### 1. Definition

Terms as used herein include the following meanings. The other terms are intended to include meanings generally used in the art.

The term “induced pluripotent stem cells” or “iPS cells” as used herein refers to artificially produced cells having pluripotency, which are not so-called embryonic stem (ES) cells, but have properties analogous to those of ES cells. The iPS cells were established for the first time by Takahashi and Yamanaka (Cell 126: 663-676 (2006)) from mouse somatic cells. Thereafter, it was demonstrated that the iPS cells could be similarly established from human somatic cells (International Publication WO2007/069666; Takahashi, K. et al., Cell 131: 861-872 (2007): Yu, J. et al., Science 318: 1917-1920 (2007); and Nakagawa, M. et al., Nat. Biotechnol. 26: 101-106 (2008)). The iPS cells have the following characteristics: they are capable of differentiating into various cells that compose an animal body (that is, a pluripotency); they are capable of maintaining semipermanent proliferation while also retaining the karyotype; and a group of genes that are generally expressed by ES cells is similarly expressed, for example. Hence, the iPS cells are cells artificially induced by reprogramming of somatic cells, having properties clearly differing from those of differentiated somatic cells.

The term “reprogramming” as used herein is also referred to as “nuclear reprogramming) and refers to a process or means during which differentiated cells are induced and converted into undifferentiated cells, particularly pluripotent cells. The reprogramming event has been originally observed by methods by which somatic cell nuclei are injected into enucleated unfertilized eggs, ES cells are fused to somatic cells, an ES cell extract is caused to penetrate into somatic cells, and the like. According to the reprogramming in the present invention, the reprogramming of somatic cells into iPS cells can occur independently of germ-line cells such as ova (or eggs), oocytes, and ES cells.

Factors that enable such reprogramming are reprogramming factors. Reprogramming factors as used herein include proteins, nucleic acids, or combinations thereof. Examples of reprogramming factors reported to date include: Oct3/4, Sox2, and Klf4; Oct3/4, Klf4, and c-Myc; Oct3/4, Sox2, Klf4, and c-Myc; Oct3/4 and Sox2; Oct3/4, Sox2, and Nanog; Oct3/4, Sox2, and Lin28; and Oct3/4 and Klf4, for example. Reprogramming factors that can be used in the present invention comprise at least Oct3/4 and Nanog, but not comprise Sox2.

In addition, the same names of reprogramming factor genes such as Oct3/4, Nanog, Sox2, Klf4, c-Myc, and Lin28 are used herein regardless of types of mammals. Although such names are generally used as mouse gene names, they are used herein not only in the case of mice, but also in the cases of humans or other mammals.

In addition, Oct3/4is also referred to as Oct3, Oct4, or POU5F1, indicating the same transcription factor. Herein, they are collectively called Oct3/4.

The term “nucleic acid(s)” as used herein refers to deoxyribonucleic acid (DNA) or ribonucleic acid (RNA), such as genomic DNA, cDNA, and mRNA.

The term “somatic cell(s)”as used herein refers to any mammalian cell(s) excluding germ-line cells such as ova (or eggs) and oocytes or totipotent cells.

The term “mammal(s)” as used herein is intended to include all mammals. Examples of particularly preferable mammals include primates, rodents, and Ungulata.

### 2. Reprogramming Factors

Reprogramming factors for inducing iPS cells from somatic cells according to the present invention are reprogramming factors from mammals, comprising Oct3/4 and Nanog, or nucleic acids encoding Oct3/4 and Nanog. The reprogramming factors may further optionally comprise another reprogramming factor(s), excluding Sox2 and a nucleic acid encoding Sox2, or a cytokine(s).

A preferred example of combinations of reprogramming factors is a combination of Oct3/4 and Nanog or a combination of Oct3/4, Nanog, and Lin28. Types of mammals, from which each reprogramming factor is derived, are not limited and any mammals can be used. Examples of preferred mammals include primates (e.g., humans, monkeys, and chimpanzees), rodents (e.g., mice, rats, guinea pigs, and hamsters), Ungulata (e.g., cattle, horses, sheep, goats, and pigs), and pet animals (e.g., dogs and cats). In general, when somatic cells from a specific mammal are reprogrammed, reprogramming factors from the same mammal are preferably used. For example, when iPS cells are induced from somatic cells from a human, reprogramming factors from a human are used.

Amino acid and nucleotide sequences of the above Oct3/4, Nanog, and Lin28, respectively, are available via access to the GenBank (NCBI, U.S.A.).

Regarding Oct3/4, for example, the sequences of human Oct3/4, mouse Oct3/4, and rat Oct3/4 are registered as NM_{—}203289 or NM_{—}002701, NM_{—}013633, and NM_{—}001009178, respectively.

Regarding Nanog, for example, the sequences of human Nanog, mouse Nanog, and rat Nanog are registered as NM_{—}024865, NM_{—}028016, and NM_{—}001100781, respectively.

Regarding Lin28, for example, the sequences of human Lin28, mouse Lin28, and rat Lin28 are registered as NM_{—}024674, NM_{—}145833, and NM_{—}001109269, respectively.

As a factor analogous to Lin28, Lin28b belonging to the same Lin family is known. Hence, in addition to Lin28 or instead of Lin28, Lin28b can be used. Regarding Lin28b, for example, the sequences of human Lin28b and mouse Lin28b are registered as NM_{—}001004317 and NM_{—}001031772, respectively.

Examples of reprogramming factors other than the above factors include, but are not limited to, one or more reprogramming factors (or a group of reprogramming factors) selected from among ECAT1, and ECAT2 (also referred to as ESG1), ECAT3 (also referred to as Fbx15), ECAT5 (also referred to as Eras), ECAT7, ECAT8, and ECAT9 (also referred to as Gdf3), ECAT10 (also referred to as Sox15), ECAT15-1 (also referred to as Dppa4), ECAT15-2 (also referred to as Dppa2), Fth117, Sa114, and Rex1 (also referred to as Zfp42), Utf1, Tcl1, and Stella (also referred to as Dppa3), β-catenin (also referred to as Ctnnb1), Stat3, Grb2, c-Myc, Sox1, Sox3, N-Myc, L-Myc, Klf1, Klf2, Klf4, and Klf5 (International Publication WO2007/069666), and, FoxD3, ZNF206, Mybl2, and Otx2 (International Publication WO2008/118820). The GenBank registration numbers (humans and mice) of the sequences of these reprogramming factors are as follows.

Regarding ECAT1, the sequences of human ECAT1 and mouse ECAT1 are registered as AB211062 and AB211060, respectively.

Regarding ECAT2, the sequences of human ECAT2 and mouse ECAT2 are registered as NM_{—}001025290 and NM_{—}025274, respectively.

Regarding ECAT3, the sequences of human ECAT3 and mouse ECAT3 are registered as NM_{—}152676 and NM_{—}015798, respectively.

Regarding ECAT5, the sequences of human ECAT5 and mouse ECAT5 are registered as NM_{—}181532 and NM_{—}181548, respectively.

Regarding ECAT7, the sequences of human ECAT7 and mouse ECAT7 are registered as NM_{—}013369 and NM_{—}019448, respectively.

Regarding ECAT8, the sequences of human ECAT8 and mouse ECAT8 are registered as AB211063 and AB211061, respectively.

Regarding ECAT9, the sequences of human ECAT9 and mouse ECAT9 are registered as NM_{—}020634 and NM_{—}008108, respectively.

Regarding ECAT10, the sequences of human ECAT10 and mouse ECAT10 are registered as NM_{—}006942 and NM_{—}009235, respectively.

Regarding ECAT15-1, the sequences of human ECAT15-1 and mouse ECAT15-1 are registered as NM_{—}018189 and NM_{—}028610, respectively.

Regarding ECAT15-2, the sequences of human ECAT15-2 and mouse ECAT15-2 are registered as NM_{—}138815 and NM_{—}028615, respectively.

Regarding Fth117, the sequences of human Fth117 and mouse Fth117 are registered as NM_{—}031894 and NM_{—}031261, respectively.

Regarding Sa114, the sequences of human Sa114 and mouse Sa114 are registered as NM_{—}020436 and NM_{—}175303, respectively.

Regarding Rex1, the sequences of human Rex1 and mouse Rex1 are registered as NM_{—}174900 and NM_{—}009556, respectively.

Regarding Utf1, the sequences of human Utf1 and mouse Utf1 are registered as NM_{—}003577 and NM_{—}009482, respectively.

Regarding Tcl1, the sequences of human Tcl1 and mouse Tcl1 are registered as NM_{—}021966 and NM_{—}009337, respectively.

Regarding Stella, the sequences of human Stella and mouse Stella are registered as NM_{—}199286 and NM_{—}139218, respectively.

Regarding β-catenin, the sequences of human β-catenin and mouse β-catenin are registered as NM_{—}001904 and NM_{—}007614, respectively.

Regarding Stat3, the sequences of human Stat3 and mouse Stat3 are registered as NM_{—}139276 and NM_{—}213659, respectively.

Regarding Grb2, the sequences of human Grb2 and mouse Grb2 are registered as NM_{—}002086 and NM_{—}008163, respectively.

Regarding FoxD3, the sequences of human FoxD3 and mouse FoxD3 are registered as NM_{—}012183 and NM_{—}010425, respectively.

Regarding ZNF206, the sequences of human ZNF206 and mouse ZNF206 are registered as NM_{—}032805 and NM_{—}001033425, respectively.

Regarding Myb12, the sequences of human Myb12 and mouse Myb12 are registered as NM_{—}002466 and NM_{—}008652, respectively.

Regarding Otx2, the sequences of human Otx2 and mouse Otx2 are registered as NM_{—}172337 and NM_{—}144841, respectively.

Regarding c-Myc, the sequences of human c-Myc and mouse c-Myc are registered as NM_{—}002467 and NM_{—}010849, respectively.

Regarding N-Myc, the sequences of human N-Myc and mouse N-Myc are registered as NM_{—}005378 and NM_{—}008709, respectively.

Regarding L-Myc, the sequences of human L-Myc and mouse L-Myc are registered as NM_{—}001033081 and NM_{—}008506, respectively.

Regarding Sox1, the sequences of human Sox1 and mouse Sox1 are registered as NM_{—}005986 and NM_{—}009233, respectively.

Regarding Sox3, the sequences of human Sox3 and mouse Sox3 are registered as NM_{—}005634 and NM_{—}009237, respectively.

Regarding Klf1, the sequences of human Klf1 and mouse Klf1 are registered as NM_{—}006563 and NM_{—}010635, respectively.

Regarding Klf2, the sequences of human Klf2 and mouse Klf2 are registered as NM_{—}016270 and NM_{—}008452, respectively.

Regarding Klf4, the sequences of human Klf4 and mouse Klf4 are registered as NM_{—}004235 and NM_{—}010637, respectively. Regarding Klf5, the sequences of human Klf5 and mouse Klf5 are registered as NM_{—}001730 and NM_{—}009769, respectively.

A system for inducing iPS cells in the method of the present invention may comprise at least one type of cytokine from a mammal, such as a human, as exemplified below in combination with the above reprogramming factors. Examples of cytokines include bFGF (basic Fibroblast Growth Factor), SCF (Stem Cell Factor), TERT (teromerase reverse transcriptase), SV40 Large T, HPV16 (human papillomavirus type 16) E6 or E7, and Bmi1 (Polycomb gene product).

According to the method of the present invention, a reprogramming factor can be used in the form of either a protein (or a polypeptide) or a nucleic acid (or a polynucleotide). Such a protein or nucleic acid can be prepared using conventional techniques such as PCR (polymerase chain reaction) techniques, gene recombination techniques, or the like.

Generally, a nucleic acid can be amplified using PCR techniques from a genomic library or cDNA library from mammalian cells containing the nucleic acid. For amplification, sense and antisense primers are prepared based on the polynucleotide sequence of the nucleic acid. In this case, primers are designed so as to anneal at each end of the ORF (open reading frame) sequence of a nucleic acid as a reprogramming factor. Generally, the size of a primer is a length ranging from approximately 17-30 nucleotides and preferably ranging from approximately 20-26 nucleotides. PCR comprises performing approximately 20-40 cycles, each consisting of a denaturation step, an annealing step, and an extension step. The denaturation step is to denature and dissociate double-stranded DNA into single-stranded DNAs (e.g., about 94-96° C. for about 30 sec to about 5 min). The annealing step is to perform annealing (that is, attaching) a sense primer or an antisense primer to each single-stranded DNA as a template (e.g., about 50-65° C. for about 30 sec to 1 min). The extension step is to extend the sense and antisense primers using single-stranded DNA as a template, so as to synthesize DNA strands (e.g., about 72° C. for about 30 sec to 10 min). Before the above cycles are performed, pretreatment may be performed at about 94-96° C. for about 2-7 min. Also, after completion of the above cycles, posttreatment may be performed at about 72° C. for about 5-10 min. As a buffer for PCR, PCR buffer (containing MgCl₂) is used. Upon reaction, primers, a thermostable DNA polymerase enzyme (e.g., Tali DNA polymerase), 4 types of deoxynucleotides (dNTPs (N=A, T, C and G)) are added to the buffer. Normally, the amount of a reaction solution ranges from approximately 10 to 100 The PCR techniques are as described in Saiki, R. K. et al., Science 230: 1350-1354 (1985), Erlich, H. A. et al., Science 252: 1643-1651 (1991); and Hughes, S. and Moody, A., PCR (Methods Express), Scion Publishing (2007), for example.

A cDNA library from mammalian cells can be prepared by extracting total mRNA from tissues or cells (including somatic cells, stem cells, or embryonic stem cells), synthesizing first strand DNA from total mRNA by a reverse transcription (RT)-PCR technique using oligo dT primers, synthesizing second strand DNA using DNA synthase I, DNA ligase, and RNaseH, blunt-ending the resultants using T4 DNA synthase, ligating an EcoR I adaptor, phosphorylating cDNA, and then ligating the cDNA to a phage vector (e.g., λgt11).

A genomic library derived from mammalian cells can be prepared by extracting genomic DNA from tissues or cells, partially digesting the resultants with an appropriate restriction enzyme (e.g., Sau3A), collecting approximately 40-kb DNA fragments by sucrose density-gradient centrifugation, and then ligating the DNA fragment to a vector such as a cosmid.

Gene recombination techniques are applied to the thus generated nucleic acid as a reprogramming factor by PCR amplification as described above, so that a vector containing the nucleic acid or a protein that is encoded by the nucleic acid can be prepared. A nucleic acid (particularly, cDNA) is inserted in a double-stranded form into a vector and can be regulated by an appropriate regulatory sequence so that a foreign nucleic acid can be expressed. Examples of a vector include plasmids, phages, cosmids, viruses (e.g., retrovirus, lentivirus, adenovirus, adeno-associated virus, and baculovirus), and artificial chromosomes (e.g., HAC, BAC, PAC, and YAC).

The regulatory sequence appropriately contains a promoter, an enhancer, a terminator, a polyadenylation site, a ribosomal binding site, a replication origin, and the like. In particular, examples of a promoter include, but are not limited to, an Oct3/4 promoter, a human cytomegalovirus (CMV) promoter, an adenovirus late promoter, a vaccinia virus 7.5K promoter, an SV40 promoter, a polyhedrin promoter, a metallothionein promoter, a cauliflower mosaic virus promoter, a tobacco mosaic virus promoter, and a glycolytic enzyme promoter. Also, a promoter can be a tissue-specific promoter, an inducible promoter, or a constitutive promoter.

Furthermore, if necessary, a vector as used herein may further contain a selection marker. Examples of a selection marker include drug resistance genes (e.g., G418, a neomycin resistance gene, and a puromycin resistance gene), reporter genes (e.g., GFP (green fluorescence protein), GUS (β-gluclonidase), and FLAG), negative selection markers (e.g., a thymidine kinase (TK) gene and a diphtheria toxin (DT) gene). In addition, a vector used herein may further contain the sequence of a multicloning site having a plurality of restriction enzyme recognition sites to facilitate insertion of a foreign nucleic acid, the sequence of IRES (internal ribosomal entry site), and the like.

Proteins used as reprogramming factors in the method of the present invention can be obtained by the method that comprises introducing an expression vector containing DNA that encodes such a protein (a signal sequence-containing precursor protein or a mature protein) into prokaryotic cells or eukaryotic cells as host cells such as bacteria (e.g., Escherichia coli, Bacillus subtilis, and the genus Pseudomonas), yeast (e.g., the genus Saccharomyces, the genus Candida, and the genus Pichia), insect cells (e.g., Sf cells), mammalian cells (e.g., CHO, NIH3T3, HEK293, COS, and BHK), or plant cells, culturing transformed or transduced host cells in an appropriate medium, and then collecting the protein of interest from the cells or medium. The protein can be collected by an appropriate combination of conventional means such as chromatography (e.g., gel filtration chromatography, ion exchange chromatography, affinity chromatography, HPLC, or FPLC), electrophoresis, isoelectric focusing, ultrafiltration, ammonium sulfate precipitation, and organic solvent precipitation.

Nucleic acids used as reprogramming factors in the method of the present invention are preferably each in the form of a vector that contains DNA encoding the above protein (which is a signal sequence-containing precursor protein or a mature protein) so that the gene can be expressed. Particularly, when such vector is used for reprogramming human somatic cells, it is desired that silencing of the above DNA takes place after completion of reprogramming or that the expression is transient. A vector used herein may be either a vector that is easily integrated into the genome of cells, such as a retrovirus or a lentivirus, or a vector that is difficult to be integrated into the genome of cells, such as adenovirus, a plasmid, or an artificial chromosome (Stadtfeld, M. et al., Science, 322, 945-949 (2008) (published online 25 September, 2008); Okita, K. et al., Science, 322, 949-953 (2008) (published online 9 October, 2008); Takahashi, K. et al., Cell 131: 861-872 (2007); and Yu, J. et al., Science 318: 1917-1920 (2007)).

General procedures for the above gene recombination techniques are described in Sambrook, J. et al., Molecular Cloning: A Laboratory Manual, 2nd edition, Cold Spring Harbor Laboratory Press (1989); Ausubel, F. M. et al., Short protocols in Molecular Biology: A Compendium Methods from Current Protocols in Molecular Biology, John Wiley & Sons (1999); and the like.

3. Substance for Improving Efficiency for Establishment of Induced Pluripotent Stem (iPS) Cells

According to the method of the present invention, iPS cells can be established from mammalian somatic cells with the use of reprogramming factors comprising Oct3/4 and Nanog, or nucleic acids encoding them, or, in addition to these reprogramming factors, another reprogramming factor(s), excluding Sox2 and a nucleic acid encoding Sox2, or a cytokine(s). It is known that when c-Myc and Klf4 that are oncogenes are used as reprogramming factors, efficiency for establishment of iPS cells is enhanced. Conversely, it is known that when either one of or both c-Myc and Klf4 are absent, efficiency for establishment of iPS cells is significantly lowered.

However, in such case, establishment of iPS cells can be accelerated by adding a substance that improves efficiency for establishment of iPS cells to an iPS cell induction system. Examples of such substances include cytokines such as a basic fibroblast growth factor (bFGF) and a stem cell factor (SCF). Furthermore, examples of such substance include low-molecular-weight compounds such as: histone deacetylase (HDAC) inhibitors, e.g., valproic acid (VPA) (Huangfu, D. et al., Nat. Biotechnol., 26 (7): 795-797 (2008)); histone methyltransferase (G9a) inhibitors, e.g., BIX-01294 (BIX) (Shi, Y. et al., Cell Stem Cell, 2: 525-528 (2008); Kubicek, S. et al., Mol. Cell 25: 473-481 (2007); Shi, Y. et al., Cell Stem Cell, 3, 568-574 (2008)); and DNA methylase (Dnmt) inhibitors, e.g., 5′-azacytidine (Huangfu, D. et al., supra). Also, a p53 inhibitor such as shRNA or siRNA against p53 or UTF1 may be introduced into cells (Yang Zhao et al., Cell Stem Cell, 3, pp 475-479, 2008). Furthermore, concerning signal transduction, activation of the Wnt signal (Marson A. et al., Cell Stem Cell, 3, pp 132-135, 2008), inhibition of mitogen-activated protein kinase and glycogen synthase kinase-3 signal transduction (Silva J. et al., PloS Biology, 6, pp 2237-2247 2008), ES cell specific miRNAs (e.g., miR-302-367 cluster (Mol. Cell Biol. Doi:10. 1128/MCB. 00398-08), miR-302 (RNA 14:1-10 (2008)), miR-291-3p, Mir-294 and miR-295 (Nat. Biotechnol. 27:459-461 (2009)), and the like can also improve an efficiency for establishment of iPS cells.

It has been inferred that the above-mentioned substances for improving efficiency for establishment exert some kind of action on both activation of genes involved in pluripotency and inactivation of genes specifically expressed in differentiated cells (Huangfu, D. et al. supra). According to the method of the present invention, reprogramming of somatic cells into iPS cells can also be accelerated with the use of a substance for improving efficiency for establishment.

Moreover, in the reprogramming steps of a somatic cell, culturing the cell under low oxygen conditions can result in improvement of an efficiency of establishment of iPS cells (Yoshida, Y. et al., Cell Stem Cell 5:237-241 (2009)). As used herein, the term “low oxygen conditions” means that the oxygen concentration in an atmosphere upon culture of cells is significantly lower than that in air. Specifically, this includes conditions of an oxygen concentration lower than the oxygen concentration in the atmosphere of 5-10% CO₂/95-90% air, for example an oxygen concentration of not more than 18%. Preferably, examples of the oxygen concentration in the atmosphere are not more than 15% (e.g., not more than 14%, 13%, 12%, or 11%), not more than 10% (e.g., not more than 9%, 8%, 7%, or 6%), or not more than 5% (e.g., not more than 4%, 3%, or 2%). Alternatively, the oxygen concentration in the atmosphere is preferably not less than 0.1% (e.g., not less than 0.2%, 0.3%, or 0.4%), not less than 0.5% (e.g., not less than 0.6%, 0.7%, 0.8%, or 0.95%), or not less than 1% (e.g., not less than 1.1%, 1.2%, 1.3%, or 1.4%).

The procedure for making such a low oxygen state in the environment of cells includes, but is not limited to, the easiest method, as a preferable example, in which cells are cultured in a CO₂ incubator capable of regulating a concentration of oxygen. Such incubators are commercially sold from a various of instrument makers (e.g., Thermo Scientific, Ikemoto Scientific Technology, Juji Field, Wakenyaku, etc.), and are usable for this purpose.

### 4. Somatic Cells

Somatic cells that can be used in the present invention are all cells from mammals, excluding germ-line cells (e.g., ova, oocytes, and embryonic stem (ES) cells) or totipotent cells, as defined above.

Mammals from which somatic cells are derived are not particularly limited and include any type of animal. Preferred mammals are selected from primates (e.g., a human, a monkey, and a chimpanzee), rodents (e.g., a mouse, a rat, a hamster, and a guinea pig), Ungulata (e.g., cattle, sheep, a goat, a horse, and a pig), and pet animals (e.g., a dog and a cat). Further preferred mammals are humans and mice.

Examples of somatic cells used herein include, but are not limited to, any of fetal somatic cells, neonate somatic cells, and mature somatic cells. Examples of the same also include any of primary cultured cells, passaged cells, and established cell lines. Examples of the same further include tissue stem cells and tissue precursor cells.

Specific examples of somatic cells include, but are not limited to, (1) tissue stem cells (somatic stem cells) such as neural stem cells, hematopoietic stem cells, mesenchymal stem cells, and dental pulp stem cells, (2) tissue precursor cells, and (3) differentiated cells such as lymphocytes, epithelial cells, endothelial cells, muscle cells, fibroblasts (e.g., skin cells), hair follicle cells, hepatocytes, gastric mucosal cells, enterocytes, splenocytes, pancreatic cells (e.g., pancreatic exocrine cells), brain cells, pneumocytes, renal cells, and skin cells.

Mammalian individuals, which are suitable as origins to obtain somatic cells, are, but are not limited to, preferably patients themselves or other persons who have an identical or substantially identical HLA type from the view points that when iPS cells obtained are used for regenerative medicine, no rejection occurs. As used herein, the term “substantially identical” with respect to HLA types means that when cells produced by inducing differentiation of iPS cells derived from somatic cells are transplanted to a patient, the HLA type matches between donor and recipient to the extent that the transplanted cells are survivable, such as the case where major HLAs (e.g., three gene loci of HLA-A, HLA-B and HLA-DR) are identical. This is applied similarly below. When iPS cells are not administered or transplanted, i.e., for example when iPS cells are used as a cell source for screening to evaluate the presence or absence of a drug sensitivity or adverse effect in patients, it is desirable to obtain somatic cells from patients themselves or other persons who have an identical gene polymorphism correlating with drug sensitivity or adverse effect.

Prior to production of iPS cells, somatic cells are obtained from a mammal such as a human. Somatic cells are cultured in a medium for culturing animal cells and then subjected to subculture if necessary, so that primary cultured cells or passage cultured cells are obtained. The thus obtained cultured cells are used for production of iPS cells.

Somatic cells can be cultured on a basal medium, such as DMEM (Dulbecco's modified Eagle medium), MEM (minimum essential medium), α-MEM (minimum essential medium alpha modification), Ham's F12, RPMI1640, or a mixture thereof, supplemented with appropriately selected substances such as serum (e.g., 10% FBS), an antibiotic(s) (e.g., penicillin and/or streptomycin), Na pyruvate, glutamine, nonessential amino acids, and L-dextrose at a temperature of about 37° C. in the presence of 5% CO₂.

5. Production of Induced Pluripotent Stem (iPS) Cells

Production of iPS cells according to the present invention can be performed by conventionally known procedures (e.g., WO2007/069666; WO2008/11820; WO2008/124133; Takahashi, K. et al., Cell 131: 861-872 (2007)) except that reprogramming factors are derived from a mammal, comprising Oct3/4 and Nanog, or nucleic acids encoding Oct3/4 and Nanog, but having no Sox2 or nucleic acid encoding Sox2.

Reprogramming factors that can be used in the present invention comprise combinations of various reprogramming factors as exemplified in Section 2 above, provided that Oct3/4 and Nanog, or nucleic acids encoding Oct3/4 and Nanog, are essential. These reprogramming factors comprise proteins or nucleic acids. A preferred form of such a nucleic acid is a vector, wherein reprogramming factors are generally ligated to the above regulatory sequence so that the expression is possible. In this case, two or more reprogramming factors comprising nucleic acids encoding Oct3/4 and Nanog are inserted into a same vector or different vectors so that expression is possible. In general, when the number of reprogramming factors ranges from approximately 2 to 4, a plurality of reprogramming factors can be tandemly linked within one vector. A promoter sequence is ligated to the 5′ side of each reprogramming factor, and a terminator sequence, a polyA sequence, and the like are ligated to the 3′ side of each reprogramming factor. A linker sequence may be placed between reprogramming factors. Preferably, a plurality of reprogramming factors are tandemly ligated so that expression is possible, thereby preparing a cassette containing them. This cassette is incorporated into a vector. For ligation of two or more reprogramming factors (or genes), any sequences which enable polycistronic expression can be employed, such as foot and mouse disease virus-derived 2A sequence (PLoS ONE3, e2532, 2008, Stem Cells 25, 1707, 2007), IRES sequence (U.S. Patent No. 4,937,190), preferably the 2A sequence.

A vector usable in the present invention can be selected from plasmids, viruses, artificial chromosomes, and the like as exemplified above. Vectors used for production of iPS cells are viral vectors such as retroviruses, lentiviruses, and adenoviruses, and plasmids. These vectors can also be similarly used in the present invention (e.g., WO2007/069666; WO2008/11820; WO2008/124133; Takahashi, K. et al., Cell 131:861-872 (2007); Stadtfeld, M. et al., Science, 322, 945-949 (2008) (online-published 25 Sep. 2008); Okita, K. et al., Science, 322, 949-953 (2008) (online-published 9 Oct. 2008)). Additionally usable vectors include episormal vector (Yu et al., Science, 324, 797-801 (2009)), transposone (e.g., piggyback transposone: Kaji, K. et al., Nature, 458:771-775 (2009); Woltjen et al., Nature, 458:766-770 (2009); sendai virus vector (J. Biol. Chem., 282:27383-27391 (2007); Japan Patent No. 3,602,058).

As other vectors, artificial chromosomes can also be used. Examples of artificial chromosomes include a human artificial chromosome (HAC), a yeast artificial chromosome (YAC), and a bacterial artificial chromosome (BAC or PAC). Among them, HAC and YAC are minichromosomes each containing a centromere, two telomeres, and a chromosome fragment. The above-mentioned cassette containing a plurality of reprogramming factors is inserted into the chromosome fragment.

Methods for introducing reprogramming factors encoding nucleic acids into cultured somatic cells from a mammal include conventional methods, such as an electroporation method, a microinjection method, a calcium phosphate method, a viral infection method, a lipofection method, and a liposome method.

Methods for introducing reprogramming factors in a form of protein into cultured mammalian somatic cells include conventional methods, such as a method using protein delivery regents, a method using fusion proteins with protein transfer domain (PTD) or cell penetrating peptide (CPP), a microinjection method, a liposome method, a lipofection method, and the like. The protein delivery regents are commercially available as: cationic lipid-based BioPOTER Protein Delivery Reagent (Gene Therapy Systems); Pro-JectTM Protein Transfection Reagent (PIERCE); ProVectin (IMGENEX); lipid-based Profect-1 (Targeting System); membrane penetrating peptide-based Penetrain Peptide (Q biogene); Chariot Kit (Active Motif); and GenomONE using HVJ envelope (from inactivated sendai virus) (Ishihara Sangyo, Japan), for example. Introduction of a protein or petide into cells can be conducted according to instructions appended to each reagent, and its general procedures are as follows. Reprogramming factors are diluted with an appropriated solvent (e.g., a buffer such as PBS or HEPES), to which a delivery regent is then added, and the mixture is incuvated for about 5-15 min at room temperature to form a complex between the reprogramming factors and the delivery regent. The complex is added to the cells in a previously exchanged serum free medium, which cells are subsequently incuvated at 37° C. for one or several hours. After that, the medium is removed and is exchanged with a serum containing medium.

Disclosed as the PTD used are: cell passing domains of the proteins such as Drosophila-derived AntP; HIV-derived TAT (Frankel, A. et al., Cell 55:1189-1193 (1988)); Green, M. & Loewenstein, P. M. Cell 55:1179-1188 (1988)); Penetratin (Derossei, D. et al., J. Biol. Chem. 269:10444-10450 (1994)); Buforin II (Park, C. B. et al., Proc. Natl. Acad. Sci. USA 97:8245-8250 (2000)); Transportan (Pooga, M. et al., FASEB J. 12:67-77 (1998); MAP (model amphipathic peptide) (Oehlke, J. et al., Biochim. Biophys. Acta. 1414:127-139(1998)); K-FGF (Lin, Z. et al., J. Biol. Chem. 270:14255-14258 (1995)); Ku70 (Sawada, M. et al., Nature Cell Biol. 5:352-357 (2003)); Prion (Lundberg, P. et al., Biochem. Biophys. Res. Commun. 299:85-90 (2002)); pVEC (Elmquist, A. et al., Exp. Cell Res. 269:237-244 (2001)); Pep-1 (Morris, M. C. et al., Nature Biotechnol. 19:1173-1176 (2001)); Pep-7 (Gao, C. et al., Bioorg. Med. Chem. 10:4057-4065 (2002)); SynB1 (Rousselle, C. et al., Mol. Pharmacol. 57:679-686 (2000)); HN-1 (Hong, F. D. & Clayman, G. L., Cancer Res. 60:6551-6556 (2000)); and HSV-derived VP22. PTD-derived CPPs include polyarginines such as 11R (Cell Stem Cell 4:381-384 (2009)) and 9R (Cell Stem Cell 4:472-476 (2009)).

A vector, which expresses fusion proteins and in which reprogramming factors-encoding cDNAs and PTD or CPP sequence have been integrated, is produced to express the fusion protein encoding DNAs, followed by collecting the fusion protein for introduction. The introduction can be conducted by the same way as above, except that a protein delivery regent is not added.

Microinjection is a method in which a protein solution is placed in a glass needle having about 1 μm tip diameter and the solution is introduced into a cell by stabbing it with the needle, whereby proteins can be introduced into a cell reliably.

The manipulation of introducing proteins can be conducted once or more, for example 1-10 times, 1-5 times, or the like, preferably by twice or more repeats, for example 3-4 times repeats. Intervals between repeats of the manipulation are 6-48 hrs for example, preferably 12-24 hrs.

For induction of iPS cells, cultured somatic cells bring into contact with the above reprogramming factors in an appropriate medium for animal cell culture, to introduce the reprogramming factors into somatic cells, whereby the cells are transformed or transduced. Examples of culture medium include, but are not limited to, media as described in Section 4 above, such as (1) a DMEM, DMEM/F12 or DME medium containing 10-15% FBS, which medium may further optionally contain LIF (leukemia inhibiting factor), penicillin/streptomycin, puromycin, L-glutamine, nonessential amino acids, β-mercaptoethanol, and the like), (2) a medium for ES cell culture containing bFGF or SCF, such as medium for mouse ES cell culture (e.g., TX-WES™, COSMO BIO) or medium for primate ES cell culture (e.g., ReproCELL™, COSMO BIO).

An example of culture procedures is as follows. Somatic cells bring into contact with reprogramming factors on a DMEM or DMEM/F12 medium containing 10% FBS at 37° C. in the presence of 5% CO₂ and are cultured for approximately 6 to 7 days. Subsequently, the cells are reseeded on feeder cells (e.g., mitomycin C-treated STO cells or SNL cells). About 10 days after contact between the somatic cells and the reprogramming factors, cells are cultured in a bFGF-containing medium for primate ES cell culture. About 30-45 days or more after the contact, iPS cell-like colonies can be formed. The culture procedures are appropriate for induction of primate iPS cells such as human iPS cells.

Alternatively, cells may be cultured using a DMEM medium containing 10% FBS (which may further optionally contain LIF, penicillin/streptomycin, puromycin, L-glutamine, nonessential amino acids, β-mercaptoethanol, and the like) on feeder cells (e.g., mitomycin C-treated STO cells or SNL cells) at 37° C. in the presence of 5% CO₂. After about 25-30 days or more, iPS cell-like colonies occur. This culture procedure is used for induction of rodent iPS cells such as mouse iPS cells.

During the above culture, medium exchange with fresh medium is performed once a day from day 2 after the start of culture. In addition, the number of somatic cells to be used for reprogramming is not limited, but ranges from approximately 5×10³ to approximately 5×10⁶ cells per culture dish (100 cm²).

The above iPS cell-like colonies on a dish are treated with a solution containing trypsin and collagenase IV (i.e., CTK solution), and the remaining colonies are seeded on the above feeder cells to similarly culture in a medium for ES cell culture, so that iPS cells or iPS cell colonies can be obtained. The iPS cells can further be passaged under similar culture conditions.

iPS cells can be identified by a test based on the properties of pluripotent cells such as ES cells. Specifically, cells are tested for properties including ES cell-specific marker gene expression, semipermanent cell proliferation potency, pluripotency (formation of three germ layers), and the like. When the cells have such properties, the cells are identified to be iPS cells (Takahashi, K. et al., Cell 131: 861-872 (2007)).

Examples of ES cell-specific marker genes include Oct3/4, Nanog, Lin28, PH34, Dnmt3b, Noda1, SSEA3, SSEA4, Tra-1-60, Tra-1-81, and Tra2-49/6F (alkaline phosphatase). Intracellular expression of the above marker genes can be detected by an RT-PCR method using primers specific to amplification of these genes. In Examples described later (“Establishment of human iPS cells”), expression of Oct3/4, Nanog, Lin28, PH34, Dnmt3b, and Noda1 genes was confirmed.

Concerning semipermanent cell proliferation potency, exponential proliferation of cells is confirmed by conducting a cell culture test for about 4-6 months or more. The colony doubling time (or population doubling time) of human iPS cells is known to be about 46.9±12.4 hours, 47.8±6.6 hours, or 43.2±11.5, for example. Hence, each of the values can be used as an indicator for proliferation potency (Takahashi, K. et al., Cell 131: 861-872 (2007)). Also, because iPS cells have high telomerase activity, the activity may be detected by a TRAP (telomeric repeat amplification protocol) method, for example. In Examples (“Establishment of human iPS cells”) described later, the produced cells exerted exponential proliferation.

Pluripotency (or the formation of three germ layers) can be confirmed based on teratoma formation and identification of each tissue (or cells) of three germ layers (endoderm, mesoderm, and ectoderm) within teratoma tissues, for example. Specifically, in the case of mouse iPS cells, cells are injected subcutaneously into a nude mouse, or in the case of human iPS cells, cells are injected into the testis of a Scid mouse, and then tumorigenesis is confirmed. Furthermore, it is confirmed that tumor tissues are composed of, for example, a cartilage tissue (or cells), a neural tube tissue (or cells), a muscle tissue (or cells), an adipose tissue (or cells), an intestine-like tissue (or cells), or the like. In Examples (“Establishment of human iPS cells”) described later, it was confirmed by histological staining that the thus generated teratomas contained neural tube tissues (ectoderm), cartilage tissues (mesoderm), intestine-like tissues (endoderm), and the like.

Based on each of the above tests, cells are confirmed to be iPS cells and then iPS cell colonies can be selected.

6. Application of Induced Pluripotent Stem (iPS) Cells

iPS cells produced by the method of the present invention have pluripotency and properties extremely analogous to those of ES cells. Hence, iPS cells can be used as alternative cells to ES cells.

Since iPS cells have pluripotency, the induction of iPS cells into various differentiated cells, precursor cells, and tissues is possible. Specifically, various differentiated cells such as nerve cells and cardiac muscle cells can be induced from iPS cells in the presence of a factor(s) such as activin A/BMP4 (bone morphogenetic protein 4) or VEGF (vascular endothelial growth factor). The thus obtained differentiated cells can be used to treat a patient by transplanting the cells into a patient, such as the patient's defective tissue, i.e., for a so-called regenerative medicine.

Moreover, iPS cells are introduced into the blastocyst of an embryo from a mammal (excluding humans) and then the resulting embryo is transplanted into the uterus of a surrogate mother of the same animal species, so that chimeric animals that have partially inherited the genotype and traits of iPS cells can be produced (WO2007/069666). Thus, alteration of a specific gene in iPS cells, elucidation of gene functions via knock-out (KO) or knock-in (KI), production of a disease model, production of a substance (e.g., protein), and the like become possible. At this time, iPS cells induced from previously gene-altered somatic cells can also be used.

Therefore, the present invention encompasses iPS cells and iPS cell populations produced by the above method, gene-altered iPS cells, and, a method for producing chimeric animals using such iPS cells, and chimeric animals, progeny animals, and the like obtained by the method, for example.

The present invention will be further described in detail by examples as follows, but the scope of the present invention is not limited by these examples.

### EXAMPLES

### Example 1

### Establishment of Human iPS Cells Using Oct3/4 and Nanog (1)

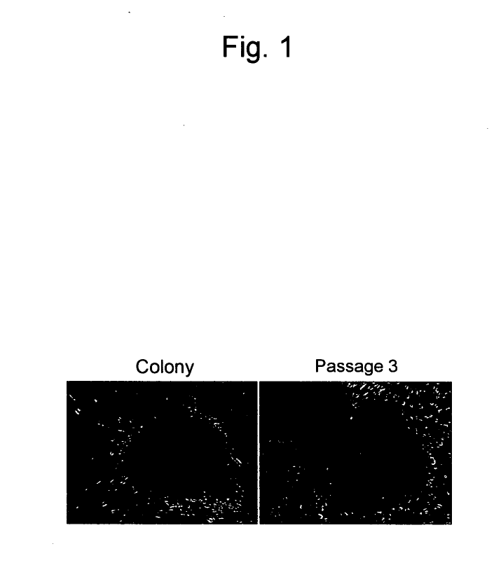

A mouse ecotropic virus receptor Slc7a1 gene was expressed by human adult dermal fibroblasts (HDF) from a 36-year-old subject according to the method described in Takahashi, K. et al., Cell, 131: 861-872 (2007) using lentivirus (pLenti6/UbC-Slc7a1). Four human genes (Oct3/4, Sox2, Nanog, and Lin28) were introduced into the cells (1×10⁵ cells/well of 6-well plates) according to the method described in Takahashi, K. et al., Cell, 131: 861-872 (2007) using a retrovirus. On day 7 after viral infection, cells were collected and then were reseeded on feeder cells (5×10⁵ cells/100-mm dish). As feeder cells, SNL cells (McMahon, A. P. & Bradley, A. Cell 62, 1073-1085 (1990)) treated with mitomycin C to cease cell division were used. From day 10 after infection, cells were cultured in medium prepared by adding 4 ng/ml recombinant human bFGF (WAKO, Japan) to medium for primate ES cell culture (ReproCELL, Japan). On day 35 after infection, two human iPS cell-like colonies appeared. Clones established from the iPS cell colonies showed human ES cell-like morphology and could maintain proliferation on feeder cells (FIG. 1).

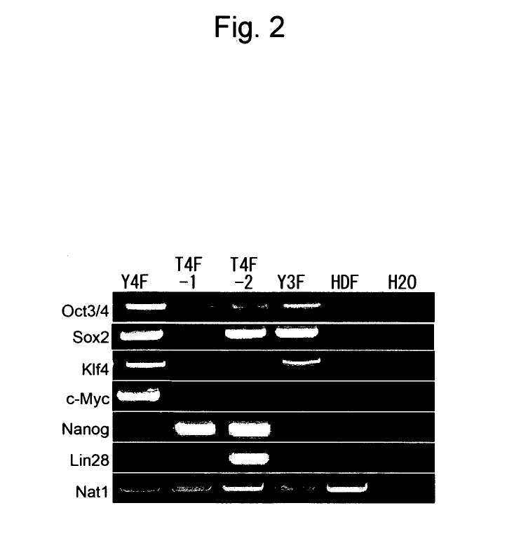

Genomic-PCR analysis was performed for the thus established human iPS cell clones according to the description of Cell, 131, 861-872 (2007), so as to examine the insertion of four genes used herein into the genome. Of the established iPS clones, one clone (T4F-2) showed insertion of all four factors into the genome, but another clone (T4F-1) showed insertion of only Oct3/4 and Nanog into the genome (FIG. 2). A retroviral vector is not stably expressed unless it is inserted into the genome. Accordingly, it was considered that the clone T4F-1 had been established via expression of only Oct3/4 and Nanog.

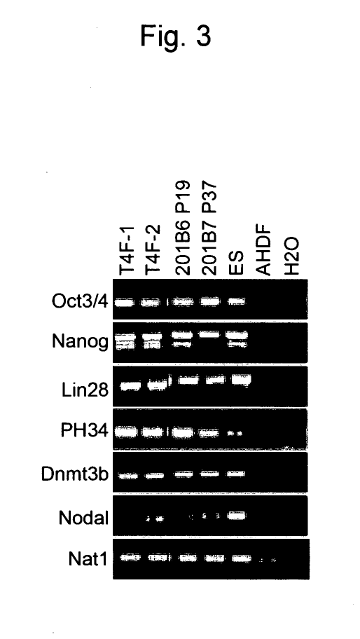

As a result of RT-PCR analysis of established clones using a Rever Tra Ace kit (Takara, Japan), these clones (T4F-1 and T4F-2) expressed human ES cell-specific marker genes, Oct3/4, Nanog, Lin28, PH34, Dnmt3b, and Nodal. The expression levels thereof were shown to be equivalent to those of human ES cells or known iPS cells (201B6 and 201B7: Takahashi, K. et al., Cell, 131: 861-872 (2007)) (FIG. 3).

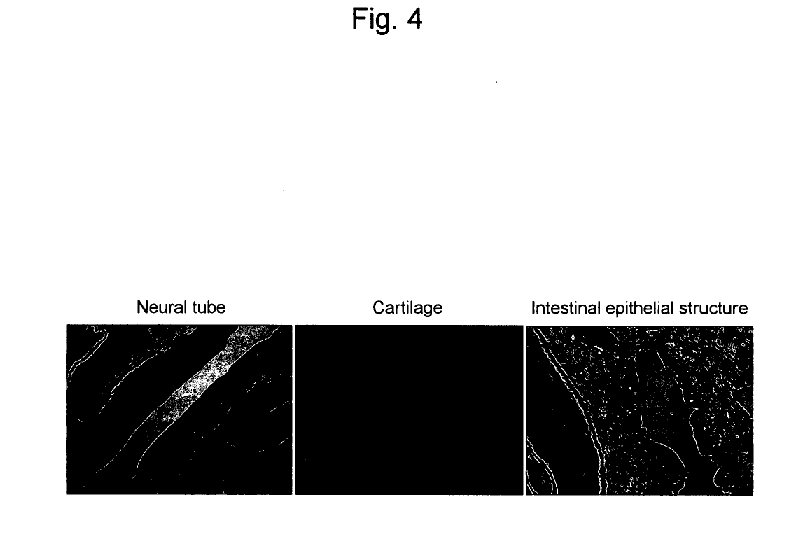

Furthermore, the clone T4F-1 was inserted into the testis of a Scid mouse to examine its pluripotency. Specifically, first, human iPS cell clone T4F-1 was cultured in medium for primate ES cell culture (ReproCELL, COSMO BIO, both Japan) containing recombinant human bFGF (4 ng/ml) and an Rho kinase inhibitor Y-27632 (10 μM). One hour later, cells were treated with collagen IV and then collected. Cells were then centrifuged, collected, and suspended in DMEM/F12 containing Y-27632 (10 μM). Confluent cells (100-mm dish) were injected in an amount ¼ into the testis of a Scid mouse. Two to 3 months later, tumors were cut into pieces and then fixed with a PBS buffer containing 4% formaldehyde. Paraffin-embedded tissues were sliced and then stained with hematoxylin-eosin. The results are shown in FIG. 4. When histologically observed, the tumor was found to be composed of a plurality of types of cells and showed differentiation into three germ layers such as neural tube tissue, cartilage tissue, and intestine-like epithelial structure. Thus, pluripotency of iPS cells was demonstrated (FIG. 4).

According to the above results, it was confirmed that clones established via expression of only Oct3/4 and Nanog were iPS cells.

### Example 2

### Establishment of Human iPS Cells Using Oct3/4 and Nanog (2)

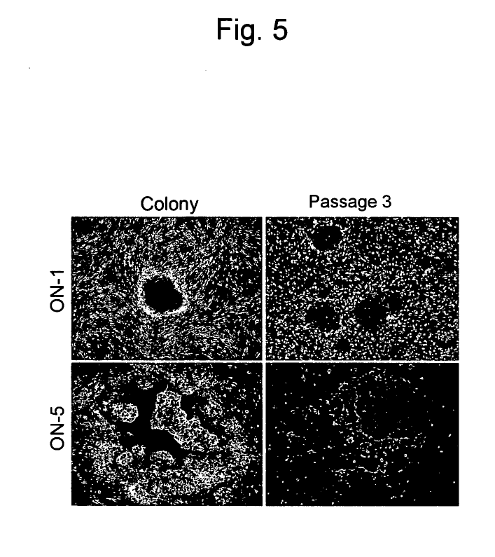

It was examined whether iPS cells could be established by the introduction of only Oct3/4 and Nanog. In a manner similar to the above, only Oct3/4 and Nanog were introduced into human adult dermal fibroblasts from a 36-year-old subject caused to express Slc7a1 using a retrovirus. On day 7 after infection, cells were reseeded on feeder cells. From day 10 after infection, cells were cultured in a medium, which was prepared by adding 4 ng/ml recombinant human bFGF (WAKO, Japan) to the medium for primate ES cell culture (ReproCELL, Japan). On day 45 after infection, one human iPS-like colony appeared (ON-1). Further four colonies (ON-2 ON-4 ON-5 ON-6) that were not human iPS-like colonies were isolated and then cultured. As a result, two (ON-5 and ON-6) out of the four clones were altered to show human ES-like morphology. The other two clones showed non ES-like morphology (ON-2 and ON-4). On day 47, the culture dishes on which iPS induction was performed were treated with a CTK solution (containing 2.5% trypsin 5 ml, 1 mg/ml collagenase IV 5 ml, 0.1 M CaCl₂ 0.5 ml, and KSR 10 ml, in 30 ml of sterilized water) and then washed with PBS. As a result, several colonies remained on the culture dishes. They were scraped together using a cell scraper, seeded on feeder cells, and then cultured. Human ES-like colonies were obtained (ON-3). The four thus established clones (ON-1, ON-3, ON-5, and ON-6) could maintain proliferation on feeder cells. The images of ES-like colonies of the clones ON-1 and ON-5, are shown in FIG. 5.

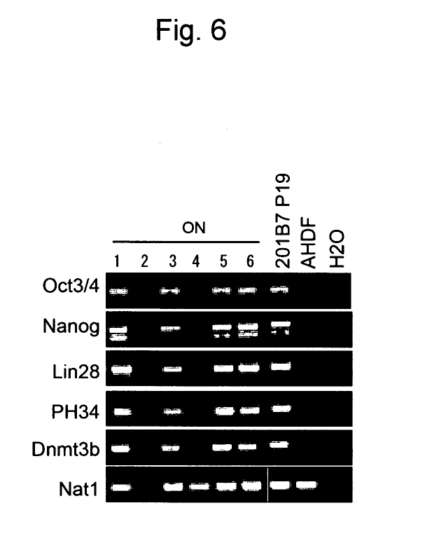

RT-PCR analysis was performed for the thus established human iPS cell clones using a Rever Tra Ace kit (Takara, Japan). As a result, all of these clones (ON-1, ON-3, ON-5, and ON-6) expressed human ES cell-specific marker genes, i.e., Oct3/4, Nanog, Lin28, PH34, and Dnmt3b, and the expression levels thereof were equivalent to those of human ES cells or 201B7 (FIG. 6).

The above results revealed that iPS cells can be established using only Oct3/4 and Nanog.

### Example 3

### Establishment of Human iPS Cells

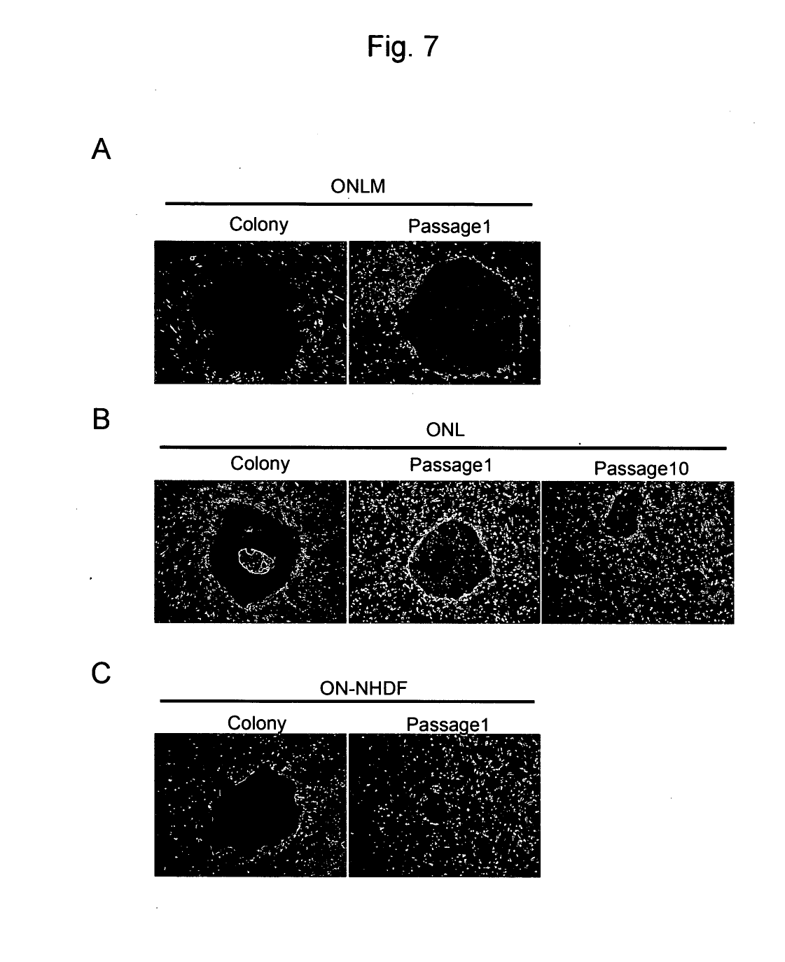

Human iPS cells (ONLM and ONL) were established (FIG. 7A and FIG. 7B) from human adult dermal fibroblasts (from a 36-year-old subject) with the same procedures as those of Example 2 except that the following reprogramming factors were used: (a). Oct3/4, Nanog, Lin28 and c-Myc; or (b) Oct3/4, Nanog, and Lin28.

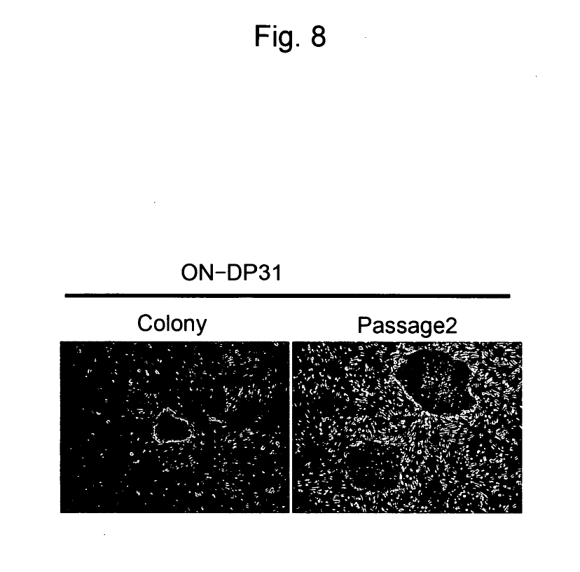

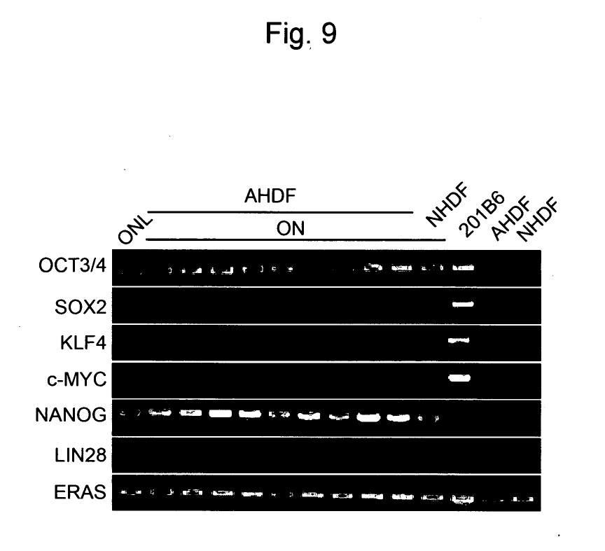

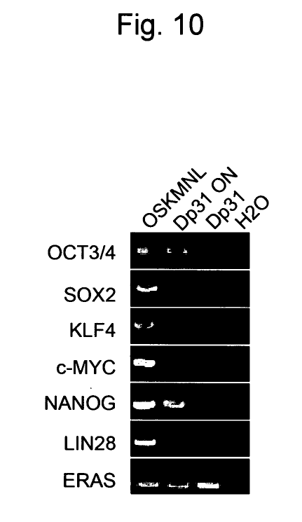

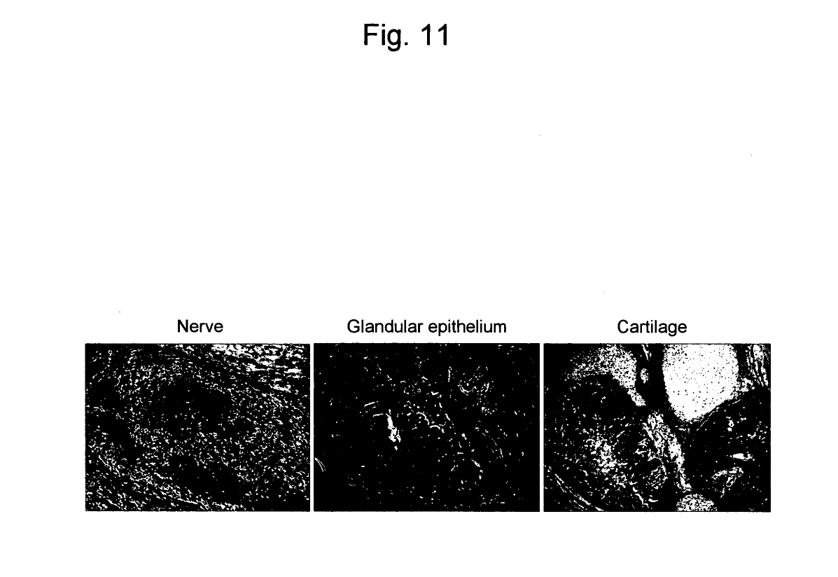

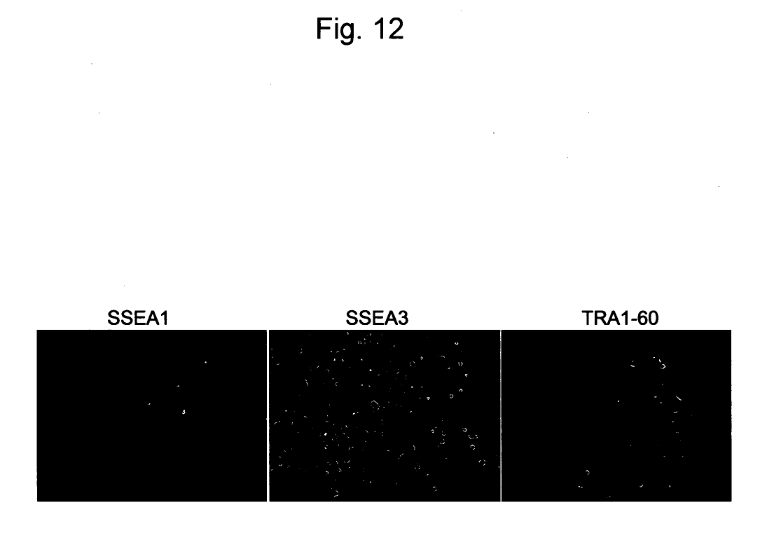

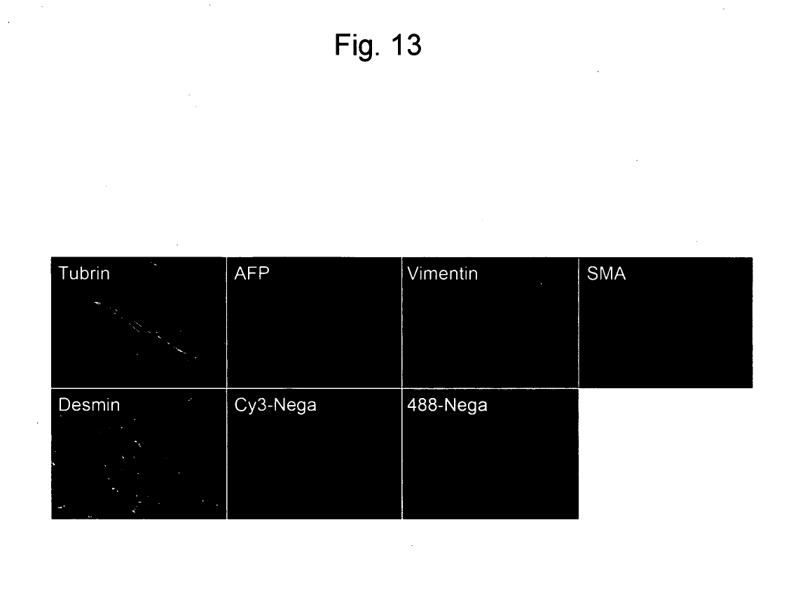

Furthermore, except for using human neonatal foreskin fibroblasts as somatic cells instead of human adult dermal fibroblasts, human iPS cells were established from cells with procedures similar to those of Example 2 with the use of only the reprogramming factors Oct3/4 and Nanog (FIG. 7C). Also, except for using dental pulp stem cells (J. Dent. Res., 87 (7): 676-681 (2008)) as somatic cells instead of human adult dermal fibroblasts, human iPS cells were established from the cells with the same procedures as those of Example 2 with the use of only the reprogramming factors, Oct3/4 and Nanog (FIG. 8).

### Example 4

### Analysis of Human iPS Cells Established Using Only Oct3/4 and Nanog

### (1) Genomic-PCR Analysis

Genomic-PCR analysis was conducted according to the description of Cell, 131, 861-872 (2007) using iPS cells from human adult dermal fibroblasts, iPS cells from human neonatal foreskin fibroblasts, and iPS cells from dental pulp stem cells, which were established in Examples 2 and 3 using only Oct3/4 and Nanog. The results are shown in FIG. 9 and FIG. 10. In all iPS cells, insertion of the thus introduced Oct3/4 gene and Nanog gene into the genome was confirmed. It was also confirmed that Sox2, Klf4, c-Myc, or Lin28 gene not used for introduction had not been inserted into the genome (FIG. 9 and FIG. 10).

### (2) Ability to Generate Teratomas

Pluripotency was examined in a manner similar to that in Example 1 using iPS cells established from human adult dermal fibroblasts using only Oct3/4 and Nanog. The results are shown in FIG. 11. A tumor was composed of a plurality of types of cells as histologically observed and showed differentiation into three germ layers such as nervous tissue, glandular epithelial tissue, and cartilage tissue. Thus, the pluripotency of iPS cells was confirmed (FIG. 11).

### (3) Expression of Undifferentiated Marker Observed Via Immunostaining

iPS cells established from human adult dermal fibroblasts using only Oct3/4 and Nanog were seeded on SNL feeders treated with mitomycin C. After 5 days of culture, cells were fixed with 4% paraformaldehyde and then treated with a PBS buffer containing 5% normal goat serum, 1% BSA, and 0.2% TritonX-100. As primary antibodies, an anti-human SSEA1 antibody (DHSB, 1:100), an anti-human SSEA3 antibody (Provided by Dr. Peter W. Andrews, 1:10), and an anti-human TRA-1-60 antibody (Provided by Dr. Peter W. Andrews, 1:50) were used. Also, as a secondary antibody, an antibody labeled with A1exa488 or Cy3 (cyanine 3) was used. The nuclei were stained with Hoechst 33342. The results are shown in FIG. 12. The thus established iPS cells expressed SSEA3 and TRA1-60. On the other hand, in the case of the anti-SSEA1 antibody, only the edges of the colonies were stained. Expression patterns similar to those of these results have been reported in the cases of human ES cells and iPS cells established in the past. It was thus demonstrated that iPS cells established with such two factors were comparable to ES cells in terms of stem cell marker expression.

### (4) In Vitro Differentiation Induction

iPS cells established from human adult dermal fibroblasts with the use of only Oct3/4 and Nanog were seeded on low-binding dishes and then subjected to 8 days of suspension culture on dishes coated with poly-hydroxyethyl methacrylate (HEMA) according to the method described in Cell, 131, 861-872 (2007). Thus, embryoid bodies (EB) were formed on plates coated with gelatin (100-mm dish). After 8 days of culture, cells were fixed with 4% paraformaldehyde and then treated with a PBS buffer containing 5% normal goat serum, 1% BSA, and 0.2% TritonX-100. Staining was performed using antibodies of each of the following: AFP (a-fetoprotein, R&D systems), which is a differentiated marker of endodermal cells; SMA (smooth muscle actin, DAKO), Desmin (NeoMarkers), and Vimentin (Santa Cruz), which are differentiated markers of mesodermal cells; and βIII-tubulin (Chemicon), which is a differentiated marker of ectodermal cells. As a secondary antibody, an antibody labeled with Alexa488 or Cy3 (cyanine 3) was used. The nuclei were stained with Hoechst 33342. The results are shown in FIG. 13.

Expression of these markers was confirmed by the staining. Moreover, it was confirmed that the established human iPS cells had an ability to differentiate into three germ layers in vitro.

### (5) Karyotype Analysis

For the established three iPS cell clones, the kryotype analysis was conducted by Nihon Gene Research Laboratories Inc., Japan. As a result, all karyotypes fell within the normal region.

Primers for Genomic-PCR and RT-PCR performed in Examples 1 to 4 above are listed in Table 1, Table 2, and the Sequence Listing (SEQ ID NOS: 1-20).

TABLE 1
Genomic-PCR

| Primer | Sequence | Seq. ID No. | Combination |
|---|---|---|---|
| hOCT3/4-S944 | CCC CAG GGC CCC ATT TTG GTA CC | 1 | pMX/L3205 |
| hSOX2-S691 | GGC ACC CCT GGC ATG GCT CTT GGC TC | 2 | pMXs-AS3200 |
| hKLF4-S1128 | ACG ATC GTG GCC CCG GAA AAG GAC C | 3 | pMX/L3205 |
| hMYC-S1011 | CAA CAA CCG AAA ATG CAC CAG CCC CAG | 4 | pMX/L3205 |
| hNANOG s | TGG AAG CTG CTG GGG AAG GCC TTA A | 5 | pMX/L3205 |
| hLin28 S502 | AGC CAT ATG GTA GCC TCA TGT CCG C | 6 | pMX/L3205 |
| pMX/L3205 | CCC TTT TTC TGG AGA CTA AAT AAA | 7 | |
| pMXs-AS3200 | TTA TCG TCG ACC ACT GTG CTG CTG | 8 | |

TABLE 2
RT-PCR

| | | Seq. |
|---|---|---|
| | | ID |
| primer | Sequence | No. |
| hOCT3/4-S1165 | GAC AGG GGG AGG GGA GGA | 9 |
| | GCT AGG | |
| hOCT3/4-AS1283 | CTT CCC TCC AAC CAG TTG | 10 |
| | CCC CAA AC | |
| Hs Nanog 3UTR S | ACA CTG GCT GAA TCC TTC | 11 |
| | CTC TCC CC | |
| Hs Nanog 3UTR AS | AGC CTC CCA ATC CCA AAC | 12 |
| | AAT ACG AA | |
| Hs Lin28 3UTR S | GGA GCA GGC AGA GTG GAG | 13 |
| | AAA GTG GG | |
| Hs Lin28 3UTR AS | CAA GGT GCA GTA TCC AAG | 14 |
| | GGA GCA AA | |
| hpH34-S40 | ATA TCC CGC CGT GGG TGA | 15 |
| | AAG TTC | |
| hpH34-AS259 | ACT CAG CCA TGG ACT GGA | 16 |
| | GCA TCC | |
| hDNMT3B-S2502 | TGC TGC TCA CAG GGC CCG | 17 |
| | ATA CTT C | |
| hDNMT3B-S2716 | TCC TTT CGA GCT CAG TGC | 18 |
| | ACC ACA AAA C | |
| hNODAL-S693 | GGG CAA GAG GCA CCG TCG | 19 |
| | ACA TCA | |
| hNODAL-AS900 | GGG ACT CGG TGG GGC TGG | 20 |
| | TAA CGT TTC | |

1. A method for producing a mammalian induced pluripotent stem cell, the method comprising:
introducing a mammal-derived reprogramming factor comprising Oct3/4 and Nanog, or nucleic acids encoding Oct3/4 and Nanog, into a mammal-derived somatic cell and thereby inducing the induced pluripotent stem cell from the somatic cell, wherein the reprogramming factor comprises neither Sox2 nor nucleic acid encoding Sox2.

2. The method according to claim 1, wherein the reprogramming factor further comprises Lin28.

3. The method according to claim 1, wherein the reprogramming factor consists of Oct3/4 and Nanog.

4. The method according to claim 1, wherein the reprogramming factor consists of Oct3/4, Nanog, and Lin28.

5. The method according to claim 1, wherein a mammal from which the reprogramming factor and somatic cell are derived the same or different from each other.

6. The method according to claim 5, wherein the mammal is a primate.

7. The method according to claim 5, wherein the mammal is a human.

8. The method according to claim 5, wherein the mammal is a mouse.

9. The method according to claim 1, wherein the nucleic acids are present and are comprised in a vector.

10. The method according to claim 1, wherein the nucleic acids are present and are integrated into a genome of the somatic cell.

11. The method according to claim 1, wherein the inducing of the induced pluripotent stem cell is carried out in the presence of a substance for improving an efficiency of establishment of the cell.

12. The method according to claim 11, wherein the substance for improving an efficiency of establishment is a histone deacetylase (HDAC) inhibitor, a histone methyltransferase (G9a) inhibitor, or a DNA methylase (Dnmt) inhibitor.

13. The method according to claim 1, wherein the reprogramming factor further comprises a nucleic acid encoding Lin28.

14. The method according to claim 1, wherein the reprogramming factor consists of nucleic acids encoding Oct3/4 and Nanog.

15. The method according to claim 1, wherein the reprogramming factor consists of nucleic acids encoding Oct3/4, Nanog, and Lin28.

16. The method according to claim 5, wherein the mammal is a rodent.

17. The method according to claim 1, wherein the nucleic acids are present and are comprised in more than one vector.

18. The method according to claim 1, wherein the nucleic acids are present and exist within the cell in a state not integrated into a genome of the somatic cell.


SEQUENCE LISTING (20 sequences)

SEQ ID NO: 1 - DNA, 23 bp, Artificial Sequence
  Description of Artificial Sequence Synthetic primer
ccccagggccccattttggtacc

SEQ ID NO: 2 - DNA, 26 bp, Artificial Sequence
  Description of Artificial Sequence Synthetic primer
ggcacccctggcatggctcttggctc

SEQ ID NO: 3 - DNA, 25 bp, Artificial Sequence
  Description of Artificial Sequence Synthetic primer
acgatcgtggccccggaaaaggacc

SEQ ID NO: 4 - DNA, 27 bp, Artificial Sequence
  Description of Artificial Sequence Synthetic primer
caacaaccgaaaatgcaccagccccag

SEQ ID NO: 5 - DNA, 25 bp, Artificial Sequence
  Description of Artificial Sequence Synthetic primer
tggaagctgctggggaaggccttaa

SEQ ID NO: 6 - DNA, 25 bp, Artificial Sequence
  Description of Artificial Sequence Synthetic primer
agccatatggtagcctcatgtccgc

SEQ ID NO: 7 - DNA, 24 bp, Artificial Sequence
  Description of Artificial Sequence Synthetic primer
ccctttttctggagactaaataaa

SEQ ID NO: 8 - DNA, 24 bp, Artificial Sequence
  Description of Artificial Sequence Synthetic primer
ttatcgtcgaccactgtgctgctg

SEQ ID NO: 9 - DNA, 24 bp, Artificial Sequence
  Description of Artificial Sequence Synthetic primer
gacagggggaggggaggagctagg

SEQ ID NO: 10 - DNA, 26 bp, Artificial Sequence
  Description of Artificial Sequence Synthetic primer
cttccctccaaccagttgccccaaac

SEQ ID NO: 11 - DNA, 26 bp, Artificial Sequence
  Description of Artificial Sequence Synthetic primer
acactggctgaatccttcctctcccc

SEQ ID NO: 12 - DNA, 26 bp, Artificial Sequence
  Description of Artificial Sequence Synthetic primer
agcctcccaatcccaaacaatacgaa

SEQ ID NO: 13 - DNA, 26 bp, Artificial Sequence
  Description of Artificial Sequence Synthetic primer
ggagcaggcagagtggagaaagtggg

SEQ ID NO: 14 - DNA, 26 bp, Artificial Sequence
  Description of Artificial Sequence Synthetic primer
caaggtgcagtatccaagggagcaaa

SEQ ID NO: 15 - DNA, 24 bp, Artificial Sequence
  Description of Artificial Sequence Synthetic primer
atatcccgccgtgggtgaaagttc

SEQ ID NO: 16 - DNA, 24 bp, Artificial Sequence
  Description of Artificial Sequence Synthetic primer
actcagccatggactggagcatcc

SEQ ID NO: 17 - DNA, 25 bp, Artificial Sequence
  Description of Artificial Sequence Synthetic primer
tgctgctcacagggcccgatacttc

SEQ ID NO: 18 - DNA, 28 bp, Artificial Sequence
  Description of Artificial Sequence Synthetic primer
tcctttcgagctcagtgcaccacaaaac

SEQ ID NO: 19 - DNA, 24 bp, Artificial Sequence
  Description of Artificial Sequence Synthetic primer
gggcaagaggcaccgtcgacatca

SEQ ID NO: 20 - DNA, 27 bp, Artificial Sequence
  Description of Artificial Sequence Synthetic primer
gggactcggtggggctggtaacgtttc